(19) United States
(12) Patent Application Publication
(10) Pub. No.: US 2003/0232386 A1
(43) Pub. Date: Dec. 18, 2003

(54) Assay conjugate and uses thereof

(76) Inventors: Dinesh O. Shah, Libertyville, IL (US); Chi-Deu Chang, Green Oaks, IL (US); Irenea V. Batac-Herman, Chicago, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/172,944

(22) Filed: Jun. 17, 2002

(51) Int. Cl. G01N 33/53; C07H 5/04; C08B 30/18; C08B 37/16; C08B 37/02; G01N 21/76; C07K 14/765

(52) U.S. Cl. 435/7.1; 436/518; 530/363; 536/18.7; 536/51; 536/46

(57) ABSTRACT

The subject invention relates to a conjugate which may be used for the detection of an analyte in a test sample. In particular, the conjugate comprises a heterophilic carrier, at least 10 label groups, an analyte-specific binding pair member (e.g., an antibody or antigen with complexes with the antigen or antibody of interest, respectively) as well as a heterophilic linker which indirectly attaches the acridinium-containing compounds to the analyte-specific binding pair member.

### BACKGROUND OF THE INVENTION

1. Technical Field

The subject invention relates to a conjugate that may be used for the detection of an analyte in a test sample. In particular, the conjugate comprises at least 10 signal-generating groups or labels, a hydrophilic carrier, a heterobifunctional linker, and an analyte-specific binding pair member (e.g., an antibody or antigen which complexes with the antigen or antibody of interest, respectively).

2. Background Information

Many methodologies have been developed to achieve highly sensitive immunoassays which detect either antigens or antibodies. Such methods include, for example, increasing a signal produced by a signal-generating compound such as a label, or reducing background interference. Immunoassays which specifically employ chemiluminescent labels as the signal-generating compound are known. For example, use of acridinium compounds as labels for immunoassays and subsequent generation of short-lived chemiluminescence signals from these labels has been described by I. Weeks in “Acridinium Esters as Highly Specific Activity Labels in Immunoassays,” Clin. Chemistry 19:1474-1478 (1984). The use of stable acridinium sulfonamide esters has been described in U.S. Pat. No. 5,468,646.

The generation of luminescent signals has been described as resulting from action of enzymes or nucleophilic agents on dioxetane compounds containing a polyclclic alkylene having at least two fused rings such as, for example, adamantine, camphorane, norbornane (see, e.g., U.S. Pat. No, 4,931,223 and U.S. Pat. No. 5,068,339). Chemiluminescent electron-rich aryl-substituted 1,2-dioxetane compounds are disclosed in which the aryl group is poly-substituted with suitable electron-donating groups such that the light-emitting pattern of the molecule results in a very high luminescent count, thus providing for a sensitive and precise assay for haptens, analytes, polynucleotides and the like. These substituted aryl-containing 1,2-dioxetane compounds can be used as direct labels in an immunoassay or when derivatized with an appropriate leaving group, chemiluminescent signals can be generated by an enzyme or a chemical. The use of stable 1,2-dioxetane compounds has been described in U.S. Pat. No. 6,001,659.

Conjugates having chemiluminescent labels are generally prepared by direct labeling of antigens or antibodies with the label. However, there are certain limitations in the direct labeling of antigens or antibodies: a) a conjugate with a high incorporation ratio of label to an antigen or antibody shows high background or more nonspecific binding and b) the label may be attached randomly at or close to an key epitope on the antigen or the binding site of the antibody that blocks the access between the analyte and analyte-specific binding pair member. Both factors compromise the assay sensitivity. Hence, conjugates made from direct labeling are mostly incorporated with 2 to 3 labels per conjugate and rarely beyond 6 labels per conjugate to perform optimally.

Blockage of antibody binding sites by a random linking reaction compromises antibody activity with the conjugate (see e.g. Beniarz et al., Bioconjugate Chem. 7, 88-95 (1996)). Methods to avoid blockage of the antibody binding site have been attempted by conjugation through the carbohydrate moieties on IgG or IgM (see, e.g., the “Fc site-specific conjugation” described by Hussain et al., Bioconjugate Chem. 5, 482-490 (1994) and “carbohydate-directed conjugation” described by J. Zaza et al. in Bioconjugate Chem. 6, 367-372 (1995)). Disadvantages with these carbohydrate-directed conjugations are, for example, complex chemical reactions and low yields of conjugates.

Methods of enhancing and amplifying the chemiluminescent signal generated in an immunoassay are known in the art. The use of a signal enhancer such as avidin-biotin also is known. For example, U.S. Pat. No. 4,228,237 describes the use of a biotin-labeled specific binding substance for a ligand, in a method which also employs an enzyme labeled with avidin. Further, the use of a biotin-anti-biotin system is described in International Patent Publication No. WO92/08979. Also, U.S. Pat. No. 4,927,769 describes a method of enhancing the chemiluminescent signal generated from acridinium-ester labeled conjugates by the addition of surfactants. Additionally, U.S. Pat. No. 4,959,182 describes a method for amplifying the chemiluminescent signal generated from alkaline phosphatase-catalyzed 1,2-dioxetanes by the addition of a surfactant and a fluorescent compound attached to it. (Amplification strategies to increase the signal in immunoassays have been reviewed by L. J. Kricka and D. Wild “Signal Generation and Detection Systems” in The Immunoassay Handbook 2^{nd} Ed., Ed. D. Wild, pps. 159-176 (Nature Publishing Group, N.Y. 2001).) Approaches to incorporate many copies of luminogenic groups in conjugates to amplify luminescent signals have been described. For example, U.S. Pat. No. 5,656,500 and U.S. Pat. No. 5,656,426 use liposomes to encapsulate many acridinium molecules and to couple to specific binding substances for amplifying chemiluminescent signals in assays. However, acridinium leakage from the liposomes is a significant disadvantage. Another method describes the use of acridinium compounds having a plurality of luminegenic groups for labeling a specific binding substance in order to achieve high luminescence efficiency in assays (see U.S. Pat. No. 8,078,502). Conjugates prepared from such compounds are stable but still suffer from low incorporation of only 2 to 5 luminogenic groups per conjugate.

In view of the above, no methods are currently known for the preparation of a stable conjugate with 10 or more copies of a label for the generation of a high and specific signal. Such conjugates, such as those of the present invention, should be extremely useful in the detection of a very small amount of analyte (in a pg/ml concentration or less) such as core antigen of hepatitis C virus (HCV) or human immunodeficiency virus (HIV), at a very early stage of infection.

All U.S. patents and publications referred to herein are hereby incorporated in their entirety by reference.

### SUMMARY OF THE INVENTION

The subject invention encompasses a conjugate comprising: a) a hydrophilic carrier, b) at least 10 signal-generating groups or labels, c) an analyte-specific binding pair member and d) a linker. The hydrophilic carrier may be, for example, a polypeptide (e.g., BSA), a polysaccharide (e.g., derivatized dextran or cylodextran) or a polyoxyethylene amine. The signal-generating groups, of which there must be at least 10, may be, for example, luminogens (e.g., acridinium-containing compounds such as acridinum ester and acridinium sulfonamide, a phenanthridinium and a 1,2-dioxetane luminol), chromogens, fluorophores, fluorogens, or radioisotopes. Furthermore, they may be the same element or a mixture thereof (e.g., 10 acridinium esters or a mixture of several acridinium-containing compounds and several phenanthridium compounds, etc.). The analyte-specific binding pair member may be, for example, an antigen, an antibody or a fragment of the antigen or antibody (e.g., Fab, Fab′ or F(ab′)₂ having the same binding activity as the antigen or antibody.

The present invention also includes a method of detecting an analyte in a test sample comprising the steps of: a) contacting a conjugate comprising: 1) at least 10 signal-generating groups; 2) an analyte-specific binding pair member; 3) a hydrophilic carrier and 4) a linker, with the test sample for a time and under conditions sufficient to form conjugate/analyte complexes; and b) determining presence of said analyte in the test sample by detecting generation of a signal produced by the at least 10 signal-generating groups of the conjugate. The analyte may be, for example, a virus, a bacterium, a parasite, a fungus, a fragment, of the virus, parasite, bacterium or fungus, an antibody to the virus, bacterium, parasite or fungus, and a fragment of the antibody to the virus, bacterium, parasite and fungus. The elements of the conjugate may be as described above.

Additionally, the present invention encompasses a method of detecting an analyte in a test sample comprising the steps of: (a) contacting the test sample with a first analyte-specific binding pair member for a time and under conditions sufficient to form analyte/first analyte-specific binding pair complexes; (b) contacting the resulting complexes of (a) with a conjugate comprising: 1) a hydrophilic carrier; 2) at least 10 signal-generating groups; 3) a second analyte-specific binding pair member and 4) a linker for a time and under conditions sufficient to form analyte/first analyte-specific binding pair member/conjugate complexes; and (c) determining the presence of the analyte in the test sample by detecting generation of a signal produced by the at least 10 signal-generating groups of the conjugate. The analyte may be as described above as well as the conjugate.

Furthermore, the present invention includes a kit for detection of an analyte in a test sample, wherein the kit comprises a conjugate, wherein the conjugate comprises: 1) a hydrophilic carrier, 2) at least 10 signal-generating groups, 3) an analyte-specific binding pair member and 4) a linker.

Also, the present invention encompasses a method of forming a conjugate comprising the steps of (a) incorporating into a hydrophilic carrier at least 10 signal-generating groups; (b) attaching a linker to said resulting product of step (a); and (c) conjugating an analyte-specific binding pair member to said attached linker of step (b) in order to form said conjugate. Again, the elements of the conjugate may be as described above.

### BRIEF DESCRIPTION OF THE DRAWINGS

FIG. 1 illustrates the methods by which one may create the conjugate of the present invention.

### DETAILED DESCRIPTION OF THE INVENTION

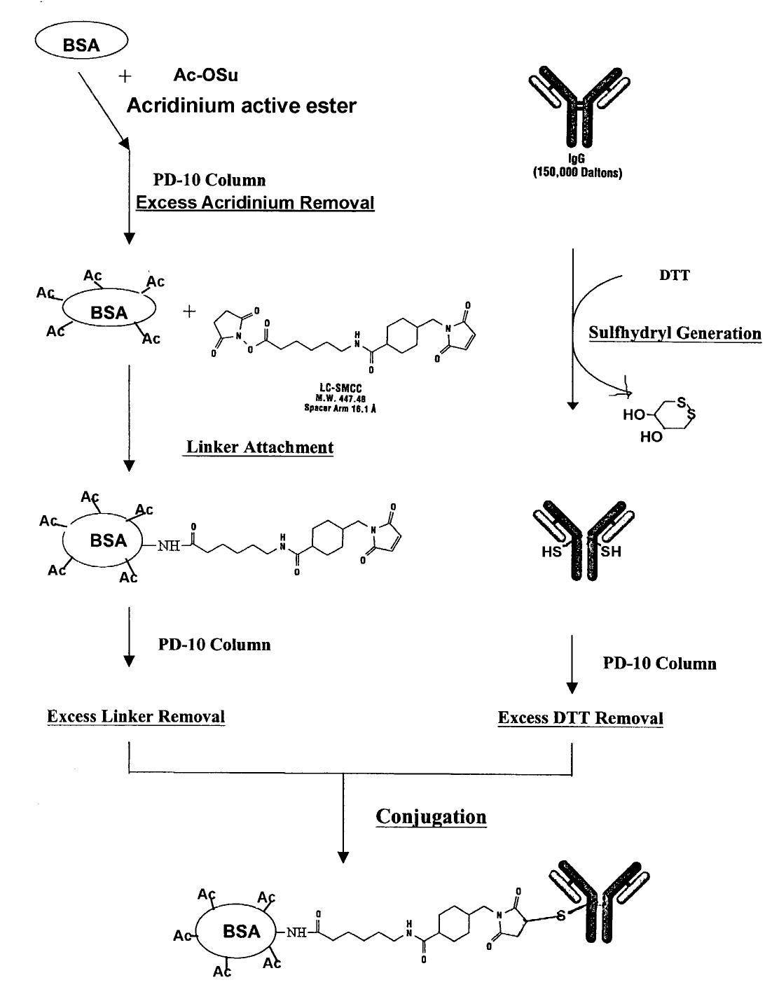

The subject invention relates to a conjugate, methods of preparing the conjugate and to methods of using the conjugate. The conjugate comprises four elements. More specifically, the conjugate comprises: 1) a hydrophilic carrier, 2) at least 10 copies of the same or different labels or signal-generating groups, 3) a linker, and 4) an analyte-specific binding pair member. The hydrophilic carrier is incorporated with preferably at least 10 copies, more preferably at least 20 copies, even more preferably at least 30 copies, and most preferably at least 40 copies of the same signal-generating group or label or different signal-generating groups or labels. (All integers between 10 and 40 are also considered to fall within the scope of the present invention.) The labels are directly coupled to the hydrophilic carrier and indirectly linked to the analyte-specific binding pair member through the carrier and the heterobifunctional linker (see FIG. 1). The hydrophilic carrier of the conjugate is attached or linked to an analyte-specific binding pair member (i.e., antigen or antibody) by a heterobifunctional linker. The conjugate amplifies the signal generated by the label(s) which indicates detection of an analyte in a test sample. In particular, the conjugate of the present invention has the ability to enhance the sensitivity for the analyte of interest in the test sample.

The “label” or “signal-generating compound” is itself detectable or may be reacted with one or more additional compounds to generate a detectable product. Examples of signal-generating compounds include chromogens, radioisotopes (e.g., I-125, I-131, P-32, H-3, S-35 and C-14), chemiluminescent compounds (e.g., acridinium-containing compounds), fluorophores, time-resolved fluorogens, luminogens, and particles (visible or fluorescent). Enzymes (e.g., alkaline phosphatase or horseradish peroxidase) may also be used in the generation of a detectable signal. Other detection systems such as time-resolved fluorescence, internal-reflection fluorescence, amplification (e.g., polymerase chain reaction) and Raman spectroscopy are also useful. The label or signal-generating group of the present invention is preferably a luminogenic group. The same or different lumogenic or chemiluminescent groups may be utilized. The luminogenic groups for signal generation may be selected from, for example, acridinium esters, acridinium sulfonamides, phenanthridiniums, 1,2-dioxetanes and luminol. A preferred luminogenic compound is an acridinium sulfonamide, as described in U.S. Pat. No. 5,468,646. These compounds are stable under normal storage conditions but can be triggered chemically to emit light with high quantum yields.

The “hydrophilic carrier”, which is used to alleviate hydrophobic non-specific binding, may be selected from, for example, polypeptides, proteins (e.g., bovine serum albumin (BSA)), polyoxyethylene amines and polysaccharides (e.g., derivatized cyclodextran and dextran). The carrier should be: 1) highly functionalized with groups, such as, for example, amines, capable of attaching many labels and a subsequent coupling reaction to the analyte-specific binding pair member, 2) highly water soluble, and 3) neutral or negatively charged. The latter two physical properties enhance solubility and reduce non-specific binding of the conjugate. A preferred hydrophilic carrier is bovine serum albumin (BSA). The carrier couples to the analyte-specific binding substance or member at its hinge, between the two heavy chains, in the case of an antibody by use of the linker arm.

Due to the limitations of increased non-specific binding and decreased antibody activity, conjugates prepared from direct labeling rarely have an incorporation ratio of label to antibody higher than 6 for optimal assay performance. However, a novel conjugate is described herein which carries at least 10 luminogenic groups per analyte-specific binding pair member (e.g., antibody or antigen) and, unexpectedly, gives equivalent or less background but significantly better responses to a positive control (e.g., HCV core antigen positive control) when compared to the control conjugate prepared from direct labeling (with an optimal incorporation of 4-6 luminogenic groups per antibody).

There are several methods by which one may prepare the conjugate of the present invention. In a preferred method, the hydrophilic carrier (e.g., BSA) is initially incorporated with multiple copies of the luminogenic group or groups (e.g., acridinium) in order to form a compound (see FIG. 1). The resulting product (e.g., (luminogenic group)x-hydrophilic carrier, for example, (Acridinium)x-BSA)) is then activated with a heterobifunctional linker. A group (e.g., maleimide) on the activated (luminogenic group)x-hydrophilic carrier) compound, for example, (Acridinium)x-BSA, is then used to react with a group, for example, a sulfhydryl group on an analyte-specific binding pair member (e.g., antibody or antigen) in order to form the final conjugate. (“X” refers to the average number of copies of the luminogenic group(s) that are covalently incorporated into each hydrophilic carrier molecule.)

Various heterobifunctional linkers can be utilized to link the (luminogenic group)x-hydrophilic carrier) compound to the analyte-specific binding pair member (e.g., antibody or antigen.) Such heterobifunctional linkers, especially maleimide active ester linkers, are particularly preferred, in that they react chemoselectively with amines on the first molecule and then conjugate to a thiol-containing second molecule in a highly controlled manner, at physiological pH. The heterobifunctional linker can be selected from, for example, succinimidyl 4-(N-maleimidomethyl)-cyclohexane-1-carboxylate, Succinimidyl 4-(N-maleimidomethyl)-cyclohexane-1-carboxy-6-amidocaporate, extended long chain linker (see U.S. Pat. No. 4,994,385), and the like.

Further, one may use sulfhydryl groups generated from the reduction of the disulfide bond at the hinge of the antibody heavy chains in the case where the analyte-specific binding substance is an antibody. The conjugation process avoids the blockage of antibody binding sites with the conjugate to allow better sensitivity. Disulfide bonds between inter-heavy chains at the hinge of the antibody are readily reduced by, for example, dithiothreitol (DTT) (5 to 50 mM) to sulfhydryl groups in a short time (i.e., within 30 minutes) at ambient temperature. After removal of excess DTT by, for example, gel-filtration, the sulfhydryl group reacts selectively to the maleimide group on the activated (luminogenic group)x-hydrophilic carrier (e.g., (Acridinium)x-BSA) which results in the formation of the conjugate in an efficient manner. The antibody may be, for example, whole IgM, IgG or (Fab′)₂.

When an antigen is the analyte-specific binding pair member and must be attached to the (luminogenic group)x-hydrophilic carrier) compound (e.g., (Acridinium)x-BSA), sulfhydryl groups may be generated on the antigen in several manners. For example, the sulfhydryl groups can be produced from a disulfide bond. For example, one may use reducing agents such as DTT or cysteine 2-mercapto-ethyl-amine (for antigens that comprise a disulfide bond). Or, one may use Traut's Reagent, 2-iminothiolane HCl, (Pierce Chemical, Rockford, Ill.) in order to introduce the sulfhydryl groups. Again, the purpose of the sulfhydryl groups is to react with a maleimide group on the activated (luminogenic group)x-hydrophilic carrier (e.g., (Acridinium)x-BSA).

The conjugate of the present invention has many uses. For example, it may be used in the detection of an analyte of interest in a test sample. In particular, the present invention encompasses a method for determining the presence of an analyte in a test sample by detection of the presence of a specific luminescent signal generated from a heterogeneous immunoassay. In one method, the conjugate may be contacted with the test sample in order for the conjugate (and, in particular, the analyte-specific binding pair member of the conjugate) to form complexes with the analyte of interest. When such complexes are formed, the luminogenic groups of the conjugate then generate a detectable chemiluminescent signal. Detection of the signal indicates presence of the analyte in the sample.

Another method of utilizing the conjugate in a diagnostic assay comprises initially incubating a test sample containing an analyte of interest with an analyte-specific binding pair member to form a first mixture for a time and under conditions sufficient to form analyte/analyte-specific binding member pair complexes.

For purposes of the present invention, “an analyte-specific binding member” is a member of a specific binding pair. That is, two different molecules where one of the molecules, through chemical or physical means, specifically binds to the second molecule. Thus, in addition to antigen and antibody specific binding pairs of common immunoassays, other specific binding pairs can include biotin and avidin, carbohydrates and lectins, complementary nucleotide sequences, effector and receptor molecules, cofactors and enzymes, enzyme inhibitors and enzymes. An analyte-specific binding pair member can also include a combination of a conjugate (as described above) and a probe (i.e., a defined nucleic acid segment which can be used to identify a specific polynucleotide (i.e., analyte) present in the test sample). (See, e.g., U.S. Pat. No. 6,207,380 B1 for a definition of a probe as well as several other terms used herein.) Furthermore, specific binding pairs can include members that are analogs of the original specific binding members, for example, an analyte analog. Immunoreactive specific binding members include antigens, antigen fragments, antibodies and antibody fragments, both monoclonal and polyclonal, and complexes thereof, including those formed by recombinant DNA molecules. (For purposes of the present invention, a “portion” or “fragment” of an antibody is defined as a subunit of the antibody which reacts in the same manner, functionally, as the full antibody with respect to binding properties. Additionally, for purposes of the present invention, a “portion” or “fragment” of an antigen is defined as an amino acid sequence which comprises at least 3 amino acids, more preferably at least 8 amino acids, and most preferably at least 15 amino acids derived from the antigen. (A “portion” or “fragment” of a nucleotide sequence refers to a contiguous sequence of at least 6 nucleotides, preferably at least 8 nucleotides, more preferably at least 10 nucleotides and more preferably at least 15 nucleotides corresponding (i.e., identical or complementary) to a region of the specified nucleotide sequence.)

The test sample may be, for example, any biological fluid such as whole blood components such as red blood cells, white blood cells, platelets, serum and plasma, ascites, urine cerebrospinal fluid, as well as other constituents of the body that may contain the analyte of interest (i.e., tissue).

An analyte is the substance to be detected which may or may not be present in the test sample. The analyte can be any substance for which there exists a naturally occurring specific binding member such as an antibody or fragment thereof (e.g., IgM, IgG, Fab, Fab′, F(ab′)₂), antigen or fragment thereof, or for which a specific binding member can be prepared. Thus, an analyte is a substance that can bind to one or more specific binding pair members in an assay. The term analyte also includes any antigenic substances, haptens (i.e., a partial antigen or non-protein binding member which is capable of binding to an antibody but which is not capable of eliciting antibody formation unless coupled to a carrier protein), antibodies or combinations thereof. As a member of a specific binding pair, the analyte can be detected by means of naturally occurring specific binding partners (i.e., pairs) such as the use of intrinsic factor protein as a member of a specific binding pair for the determination of, for example, Vitamin B12, or the use of lectin as a member of a specific binding pair for the determination of a carbohydrate. The analyte can include a protein, a peptide, an amino acid, a hormone, a steroid, a vitamin, a drug, a bacterium, a yeast, a nucleic acid sequence, any entity detected in a clinical chemistry assay, a virus (e.g., Hepatitis B Virus, Hepatitis C Virus, Human Immunodeficiency Virus, and HTLV), a parasite, fragments of the above (e.g., epitopes) as well as metabolites of or antibodies to any of the above substances. The details for the preparation of such antibodies and the suitability for use as specific binding members are well known to those of ordinary skill in the art.

Once the analyte/analyte-specific binding member pair complexes have formed, they may be contacted with the conjugate described above. In particular, the conjugate comprises another analyte-specific binding member, a hydrophilic carrier and multiple copies of one or more types of luminogenic compounds or groups, to form a second mixture. For purposes of the present invention, a conjugate comprises at least 10 luminogenic compounds or groups to which an entity specific for the analyte (i.e., analyte-specific binding member) is covalently linked (by a linker arm) through a hydrophilic carrier.

The second mixture is then incubated for a time and under conditions sufficient to form analyte/analyte-specific binding member pair/conjugate complexes. Finally, one then determines the presence of the analyte in the test sample by measuring the presence of the detectable signal generated by the luminogenic groups.

It should be noted that initially, the analyte-specific binding pair member can be attached to a solid phase. A solid phase refers to any material which is insoluble or can be made insoluble by a subsequent reaction. The solid phase can be chosen for its intrinsic ability to attract and immobilize the capture reagent (i.e., an unlabeled specific binding member which is specific either for the analyte or for an ancillary specific binding member). Alternatively, the solid phase can retain an additional receptor which has the ability to attract and immobilize the capture reagent or analyte-specific binding member. The solid phase can also retain an additional receptor which has the ability to attract and immobilize the capture reagent. Examples of a solid phase include fiberglass, cellulose, a nylon pad, a dipstick, a test strip, polymeric or glass beads, microparticles, tubes, sheet, plates, microtiter wells, polymeric films, paper, silica gel, agarose, slides, webs, tapes, test tubes or any material which has an intrinsic charge or which can retain a charged substance. The conjugate may also be used in an assay carried out in solution.

It should be noted that the conjugate of the present invention may also be utilized in a nucleic acid detection or molecular screening assay in order to detect a particular nucleotide sequence or portion thereof (as defined above) associated with a particular viral infection (e.g., HBV, HCV, HIV, HTLV, etc.), bacterial infection, parasitic infection, etc. Thus, for such an assay, the analyte-specific binding member is a probe (or nucleic acid sequence) which hybridizes to the sequence of interest in the test sample. In particular, a “probe” is a specific oligonucleotide sequence which is complementary to a target sequence and used to hybridize to the target sequence, under the appropriate conditions. Hybridization conditions (e.g., stringent, moderately stringent, etc.) useful for probe/analyte hybridization, where the probe and analyte have a specific degree of sequence identity can be determined by one of ordinary skill in the art (see, e.g., Nucleic Acid Hybridization: A Practical Approach, eds. Hames et al., 1985, Oxford; Washington, D.C.; IRL Press); see also Sambrook et al., Molecular Cloning: A Laboratory Manual, Second Edition, 1989, Cold Spring Harbor, N.Y.).

Other assays which may use the conjugate of the present invention include, for example, fluorescence polarization (e.g., homogeneous assay in solution), indirect assays, direct assays, competitive assays, rapid tests, molecular screening assays, Western blots, inhibition assays, photocytometry and tissue staining assays (see Quinn, The Immunoassay Handbook, Second edition, Wild, ed., 2001). Such formats are readily known by those of ordinary skill in the art.

The present invention also encompasses a kit comprising the conjugate described above. Such a kit could be used to detect nucleic acid sequences, antigens, antibodies or portions thereof (e.g., haptens, epitopes, Fab fragments, etc.) resulting from, for example, viral, bacterial, fungal (e.g., yeast) and parasitic infections.

It should be noted that the above method and, in particular, the above conjugate, may be used as a substitute in currently available assays which utilize a conjugate, thereby improving 1) signal amplification upon detection of the analyte of interest and 2) sensitivity for the analyte of interest.

The present invention may be illustrated by the use of the following, non-limiting examples:

### EXAMPLE I

Preparation of (Acridinium)x-Bovine Serum Albumin

A solution of bovine serum albumin (BSA) was prepared by dissolving crystalline BSA at a concentration of 6.7 mg/ml in a phosphate buffer with 0.1% CHAPS [3-(3-cholamidopropyl dimethylamino)-1-propane sulfonate]. Two hundred and ninety ml of acridinium active ester in DMF [N,N-dimethylformamide] were added to one ml of the BSA solution in a 4 ml amber vial. The active ester was prepared from 10-(3-sulfopropyl)-N-(2-carboxyethyl)-9-acridinium carboxamide (U.S. Pat. No. 5,468,646) and N-hydroxysuccinimide, and dissolved in DMF at 4 mg/ml. The reaction vial was capped, the solution was mixed by vertex, and then placed in ambient temperature for 50-60 minutes. The solution was then transferred to a PD10 column (Amersham Biosciences, Uppsala, Sweden) for G-25 gel-filtration and eluted with the phosphate buffer with 0.1% CHAPS. The yellow peak at the front void was collected in a clean 4-ml amber vial. The absorbance of acridinylated BSA was measured at 280 and 370 nm for the estimation of protein concentration and incorporated Acridinium per BSA molecule. The concentration was calculated 1.45 mg/mi with 12.8 Acridinium per BSA.

### EXAMPLE II

Attaching Maleimide Group to (Acridinium)x-BSA

(a) Activation with 30-Atom Linker:

One ml of (Acridinium)x-BSA (from Example 1 in an amber vial) was added to 108 μl of 30-atom linker, Succinimidyl (4-tricaproamido cyclohexylmethyl) N-maleimide (U.S. Pat. No. 4,994,385) which was dissolved in DMF at 5 mM. The reaction vial was capped, the solution was mixed by vertex and then placed in ambient temperature for 60 minutes. The solution was then transferred to a PD10 column for gel-filtration and eluted with a phosphate buffer with 0.1% CHAPS and 5 mM EDTA [ethylenediaminetetraacetate]. The yellow peak containing the (Acridinium)x-BSA-30-Atom Maleimide at the front was collected in a clean 4-ml amber vial, and it was stored in an ice-bath until conjugation the same day.

(b) Activation with Long Chain SMCC:

One ml of (Acridinium)x-BSA (from Example 1 in an amber vial) was added to 108 μl of Succinimidyl 4-(N-maleimidomethyl)-cyclohexane-1-carboxy-6-amidocaporate (LC-SMCC, Pierce Chemical Co., Rockford, Ill.) which was freshly dissolved in DMF to 5 mM. The reaction vial was capped, the solution was mixed by vertex and placed in ambient temperature for 60 minutes. Then, the solution was transferred to a PD10 column for gel-filtration and eluted with a phosphate buffer with 0.1% CHAPS and EDTA. The yellow peak containing the (Acridinium)x-BSA-LC Maleimide at the front was collected in a clean 4 ml amber vial, and it was stored in a 2-8° C. refrigerator until conjugation the same day.

### EXAMPLE III

Conjugation of (Acridinium)x-BSA-30-Atom Maleimide with Reduced Mab c11-10 IgG

To 3.5 mg of monoclonal anti-HCV core antigen, clone c11-10 (Tonen Corp., Ohi-Machi, Japan) in one ml of pH 7.0 phosphate buffer with 0.1% CHAPS in an 1.5 ml-vial was added 50 μl of 0.5 M dithiothreitol, freshly dissolved in distilled water, to a concentration of 25 mM. The solution was mixed by gentle inversions and set at ambient temperature for 30 minutes. Then, the solution was transferred to a PD10 column for gel-filtration and eluted with the pH7.0 phosphate buffer with 0.1% CHAPS and 5 mM EDTA. The reduced IgG in the A280 peak was collected from the void volume in a clean 4-ml amber vial and kept on ice-bath until the conjugation.

The conjugation was started within 10 minutes after the collection of reduced antibody. Two point two ml of (Acridinium)x-BSA-30-Atom Maleimide from Example II(a) was added to 1 ml of reduced c11-10 IgG in an amber vial and mixed by gentle inversions. The vial was placed in a 2-8° C. refrigerator for 18 to 20 hours. The conjugate mixture was added to Proclin 300 (Rohm-Hass, Philadelphia, Penn.) to a 0.1% final concentration and filtered through 0.22 micron filter.

The crude conjugate was fractionated on size exclusion high pressure liquid chromatography (HPLC) with a 300×7.8 mm Bio-Sil SEC-400-5 column (Bio-Rad, Richmond, Calif.) and eluted with a phosphate saline buffer with 0.1% CHAPS. The HPLC was run on a Beckman Golden System (Beckman Coulter Inc., Palo Alto, Calif.). The flow rate was 1 ml/min and the effluent was monitored with dual channels at wavelengths of 280 and 370 nm. Each injection was 200 μl, and three fractions were collected around the peak ahead of the peak of free IgG. The absorbance of each fraction was measured at 280 and 370 nm on a Beckman DU-7 spectrophotometer (Beckman Coulter Inc., Palo Alto, Calif.) for the estimation of protein concentration and incorporated Acridinium per IgG molecule. The following table shows the estimates:

| | | IgG conc | Acridinium |
|---|---|---|---|
| | Retention Time | (microgm/ml) | Per IgG |
| Fraction-1 | 9.0 to 10.0 min. | 13 | 6.80 |
| Fraction-2 | 10.0 to 10.5 min. | 22 | 9.16 |
| Fraction-3 | 10.5 to 11.5 min. | 29 | 12.35 |

Fraction-3 with a retention time from 10.5 to 11.5 minutes contains a peak (280 and 370 nm) corresponding to a molecular weight around 220 kDa (based on reference protein standard on the HPLC elution profile), indicative of a monomeric conjugate with Acridinium-BSA and IgG in 1:1 molar ratio. Fraction-1 and 2 appeared as extended front shoulder with retention time from 9.0 to 10.5 minutes corresponding to molecular weights ranging between 300 to 670 kDa, indicative of a mixture of oligomeric conjugates in various combinations of Acridinium-BSA and IgG.

### EXAMPLE IV

Conjugation of (Acridinium)x-BSA-LC Maleimide with Reduced Mab c11-10 IgG

The reduced c11-10 IgG was prepared from 1.78 mg c11-10 IgG per ml by the same procedure described in Example III. Also, the conjugation began within 10 minutes after the collection of reduced antibody. 0.8 ml of (Acridinium)x-BSA-LC Maleimide from Example II(b) was added to 0.25 ml of reduced c11-10 IgG in an amber vial and mixed by gentle inversions. The vial was set in a 2-8° C. refrigerator for 18 to 20 hours. Proclin 300 to 0.1% was added to the conjugate mixture and filtered through 0.22 micron filter. The crude conjugate was fractionated on the SEC-HPLC as described in Example III. Each injection was 200 μl, and two fractions were collected around the peak ahead of the free IgG peak. Each fraction was measured at 280 and 370 nm and estimates of IgG concentration and incorporated Acridinium per IgG molar ratio are shown in the following table.

| | | IgG conc | Acridinium |
|---|---|---|---|
| | Retention Time | (microgm/ml) | Per IgG |
| Fraction-1 | 9.5 to 10.5 min. | 9 | 13.5 |
| Fraction-2 | 10.5 to 11.4 min. | 27 | 11.7 |

Fraction-2 with a retention time from 10.5 to 11.4 minutes contains the main peak (280 and 370 nm) corresponding to a molecular weight around 220 kDa (based on reference protein standard on the HPLC elution profile), indicative of a monomeric conjugate with Acridinium-BSA and IgG in 1:1 molar ratio. Fraction-1 is part of an extended front shoulder with retention time from 9.5 to 10.5 minutes corresponding to molecular weights ranging between 300 to 670 kDa, indicative of a mixture of oligomeric conjugates in various combinations of Acridinium-BSA and IgG.

### EXAMPLE V

Conjugation of (Acridinium)x-BSA-LC Maleimide with Mab c11-10 Fab′

The c11-10 (Fab′)₂ was prepared by pepsin digestion and purified from Superdex 200G column (Aoyagi et al., J. Clin. Microbiol. 37, p1802-1808 (1999)). The conjugation of Acridinium-BSA and c11-10 Fab′ was carried out by the same procedure described in Example III but started from 0.5 mg of c11-10 (Fab′) ₂ instead of IgG. The conjugation began within 10 minutes after the collection of c11-10 Fab′ from the DTT reduction of c11-10 (Fab′)₂. 0.8 ml of (Acridinium)x-BSA-LC Maleimide from Example II(b) was added to 0.8 ml of c11-10 Fab′ in an amber vial and mixed by gentle inversions. The vial was set in a 2-8° C. refrigerator for 18 to 20 hours. The conjugate mixture was added to Proclin 300 to 0.1% and filtered through 0.22 micron filter. The crude conjugate was fractionated on size exclusion HPLC with a 600×21 mm TSK-Gel G3,000 column (Tosoh Biosep LLC, Montgomeryville, Pa.) on a Varian ProStar System (Varian Inc., Walnut Creek, Calif.). and eluted with a phosphate saline buffer with 0.1% CHAPS. The flow rate was 3 ml/min, and the effluent was monitored with dual channels at wavelengths of 280 and 370 nm. The injection was one ml and 6 fractions, one fraction per minute, were collected from a retention time 32 to 38 minutes. The absorbance of each fraction was measured at 280 and 370 nm on a Varian Cary 50 Scan spectrophotometer (Varian Inc., Walnut Creek, Calif.) for the estimation of protein concentration and incorporated Acridinium per Fab′. Estimates of Fab′ concentration and incorporated Acridinium per Fab′ molar ratio are shown in the following table.

| | | Fab′ conc | Acridinium |
|---|---|---|---|
| | Retention Time | (microgm/ml) | Per Fab′ |
| Fraction-1 | 32 to 33 min. | 1.2 | 26.50 |
| Fraction-2 | 33 to 34 min. | 1.5 | 20.69 |
| Fraction-3 | 34 to 35 min. | 1.8 | 12.13 |
| Fraction-4 | 35 to 36 min. | 2.7 | 12.19 |
| Fraction-5 | 36 to 37 min. | 4.3 | 11.84 |
| Fraction-6 | 37 to 38 min. | 6.7 | 11.17 |

Fractions 1 and 2 with a retention time from 32 to 34 minutes contains a side peak (280 and 370 nm) corresponding a to molecular weight around 200 kDa (based on reference protein standard on the HPLC elution profile), indicative of a conjugate with Acridinium-BSA and Fab′ in 2:1 molar ratio. Fractions 5 and 6 consist of a main peak with a retention time of 36 to 38 minutes corresponding to 150 kDa (size of native IgG) which is about the size of a conjugate with Acridinium-BSA and Fab′ in 1:1 molar ratio. Fractions 3 and 4 are overlapping shoulders between the 2 peaks of conjugates; they are a mixture of both kinds, probably.

### EXAMPLE VI

Sandwich Assay for HCV Core Antigen

The assay was performed by using a single channel PRISM® instrument (Abbott Laboratories, Abbott Park, Ill.) as described in the publication “Automated Panel Analyzers—PRISM” by D. Shah and J. Stewart, (Immunoassay Handbook, 2^{nd} Edition, Ed., D. Wild, Nature Publishing, NY, N.Y.).

Briefly, 100 μl of a control or serum or plasma sample; 50 μl of specimen diluent buffer (SDB) which was a borate saline with detergent, BSA, superoxide dismutase (SOD), and 0.1% sodium azide; and 50 μl of microparticles coupled with c11-14 Mab anti-HCV core antigen (Mab from Tonen) were added to an incubation well at Station 1. The reaction tray was moved step by step to Station 4 with a lapse of 18 minutes. At Station 4, the reaction mixture was transferred to a reaction well by a transfer wash buffer which was a Tris saline with detergent and 0.1% Proclin 300. Microparticles in the reaction mixture were captured by a fibrous matrix (which allows liquid to filter down) in the reaction well. At Station 5, 50 μl of conjugate containing an acridinium labeled anti-HCV core was dispensed to the reaction well. The reaction tray was moved with a lapse of 22.3 minutes to Station 8. At Station 8, the reaction mixture in the reaction well was washed with final wash buffer that was a MES [2-N-Morpholino-ethanesulfonic acid] buffered saline with detergent and 0.1% Proclin 300. The tray was then moved to Station 9 where the trigger solution, an alkaline hydrogen peroxide, was injected to the reaction well and its signal was measured by a photo-multiplier tube. The chemiluminescence counts from each reaction well were integrated and saved in alignment with its sample ID to the batch file. Assay Controls: Negative Calibrator (NC) is a re-calcified normal human plasma tested negative for HIV, HTLV, HBsAg, HBcore and HCV. AgPC This plasma has HCV RNA at 17 million copies per ml and was tested positive with an HCVcore Ag ETA, but tested negative by all current HCVAb assays. AgPC, HCV antigen positive, was diluted from an HCVAg positive human plasma (NABI, Boca Raton, Fla.) to assess the HCVAg detectability. This plasma was tested negative for HCV antibodies but NAT (Nucleic Acids Test) positive with 17,000,000 HCV RNA copies per ml by PCR (nuclear acid polymerase chain reaction), also tested highly positive on an HCV Ag assay.

### EXAMPLE VII

Testing Conjugate of (Acridinium)x-BSA-30-Atom linked c11-10 IgG

The conjugate Fraction-2 and 3 isolated from SEC-HPLC in Example III each was diluted to 100 ng/ml in a conjugate diluent of 3-N-Morpholino-propanesulfonic acid (MOPS) buffered saline with detergent, fetal calf serum, and 0.1% Proclin 300. A c11-10 conjugate prepared from direct labeling of 10-(3-sulfopropyl)-N-(2-carboxyethyl)-9-acridinium carboxamide was used as the control. The conjugates were tested in a same run for responses to NC and AgPC. The P/N ratio with each conjugate was calculated to assess the sensitivity. The background was the average counts of 6 NC and the positive response was the average counts of AgPC in duplicates.

| | Acridinium | Average | Average | P/N |
|---|---|---|---|---|
| Conjugate | Per IgG | NC | AgPC | Ratio |
| Control | 5.6 | 88 | 438 | 4.98 |
| Acr-c11-10 IgG | | | | |
| (Acr) x-BSA-30- | | | | |
| Atom linked | | | | |
| c11-10 TgC: | | | | |
| Fraction-2 | 9.16 | 74 | 585 | 7.86 |
| Fraction-3 | 12.35 | 84 | 586 | 6.98 |

Notes: NC, negative control, was prepared from pooled negative plasma; each specimen in the pool was tested non-reactive for HBsAg and negative for antibodies to HBc, HIV, HTLV, and HCV. AgPC, HCV antigen positive, is described in Example VI. P/N is the ratio of the sample response to the negative control response in counts of chemiluminescence. The P/N values provide good assessment for the sensitivity or detectability of HCV core antigen in the assay. Comparing to the control conjugate, both conjugate Fraction-2 and 3 showed comparable NC counts but significantly better response to AgPC in counts and P/N values.

### EXAMPLE VIII

Testing Conjugate of (Acridinium)x-BSA-LC linked c11-10 IgG

The conjugate Fraction-2 isolated from SEC-HPLC in Example IV each was diluted to 100 ng/ml in a conjugate diluent of MOPS saline with detergent, fetal calf serum, and 0.1% Proclin 300. The same c11-10 conjugate with directly labeled acridinium of Example VII was used as the control. Both conjugates were tested in a same batch for responses to NC and AgPC. The P/N ratio with each conjugate was calculated to assess the sensitivity. The background was the average counts of 12 NC and the positive response was the average counts of AgPC in 4 replicates.

| | Acridinium | Average | Average | P/N |
|---|---|---|---|---|
| Conjugate | Per IgG | NC | AgPC | Ratio |
| Control | 5.6 | 86 | 641 | 7.45 |
| Acr-c11-10 IgG | | | | |
| (Acr) x-BSA-LC | | | | |
| linked c11-10 IgG | | | | |
| Fraction-1 | 13.5 | 108 | 1,454 | 13.46 |
| Fraction-2 | 11.7 | 89 | 1,702 | 19.12 |

Compared to the control conjugate, the conjugate Fraction-1 (oligomeric conjugate) showed more than double signal response to AgPC with somewhat higher NC counts while Fraction-2 (monomeric conjugate) showed even better signal response to AgPC with comparable NC counts and a 2.5-fold increase in P/N value. In other words, the monomeric conjugate provides significantly better sensitivity in detecting HCV core Ag compared to the oligomeric conjugate or the control Acr-c11-10 conjugate.

### EXAMPLE IX

Testing Conjugate of (Acridinium)x-BSA-LC Linked c11-10 Fab′

The conjugate fractions isolated from SEC-HPLC in Example V were each diluted to 50 ng/ml in a conjugate diluent of a MOPS saline with detergent, fetal calf serum, and 0.1% Proclin 300. The same c11-10 conjugate,-with directly labeled acridinium of Example VII, was used as the control. All conjugates were tested in the same batch for responses to NC and AgPC. The P/N ratio with each conjugate was calculated to assess the sensitivity. The background was the average counts of 6 NC, and the positive response was the average counts of duplicate AgPC.

| | Acridinium | Average | Average | P/N |
|---|---|---|---|---|
| Conjugate | Per Fab′ | NC | AgPC | Ratio |
| Control | 5.6 | 116 | 921 | 7.92 |
| Acr-c11-10 IgG | | | | |
| (Acr) x-BSA-LC | | | | |
| linked c11-10 | | | | |
| Fab′ | | | | |
| Fraction-2 | 20.69 | 78 | 4,047 | 52.02 |
| Fraction-4 | 12.19 | 60 | 2,298 | 38.61 |
| Fraction-6 | 11.17 | 55 | 1,567 | 28.40 |

Compared to the control conjugate, all three conjugate fractions showed significantly less NC counts but much higher responses to AgPC, with 3.6 to 6.6-fold increases in P/N values. Apparently, the conjugate with Acridinium-BSA and Fab′ in 1:1 ratio (Fraction-6) showed very good sensitivity enhancement in HCV core Ag detection. The conjugate with Acridinium-BSA and Fab′ in 2:1 molar (Fraction-2) showed the most enhanced sensitivity. In other words, this conjugate should be able to detect HCV core antigen at 1/6 or less the concentration detectable by the control Ac-c11-10 IgG conjugate in the same assay system.

1. A conjugate comprising: a) a hydrophilic carrier, b) at least 10 signal-generating groups, c) an analyte-specific binding pair member and d) a linker.

2. The conjugate of claim 1 wherein said hydrophilic carrier is selected from the group consisting of a polypeptide, a polysaccharide and a polyoxyethylene amine.

3. The conjugate of claim 2 wherein said polysaccharide is selected from the group consisting of derivatized dextran and cyclodextran.

4. The conjugate of claim 2 wherein said polypeptide is bovine serum albumin (BSA).

5. The conjugate of claim 1 wherein said at least 10 signal-generating groups are selected from the group consisting of a luminogen, a chromogen, a fluorophore, a fluorogen, and a radioisotope.

6. The conjugate of claim 5 wherein said luminogen is selected from the group consisting of an acridinium-containing compound, a phenanthridinium and a 1,2-dioxetane luminol.

7. The conjugate of claim 6 wherein said acridinium-containing compound is selected from the group consisting of an acridinium ester and an acridinium sulfonamide.

8. The conjugate of claim 5 wherein said at least 10 signal-generating groups are luminogenic groups, wherein said at least 10 luminogenic groups are the same or a mixture thereof.

9. The conjugate of claim 1 wherein said analyte-specific binding pair member is selected from the group consisting of an antigen, an antibody and a fragment of said antigen or antibody having the same binding activity as said antigen or said antibody.

10. The conjugate of claim 9 wherein said fragment of said antibody is selected from the group consisting of wherein said fragment of said antibody is selected from the group consisting of Fab, Fab′ and F(ab′)₂.

11. The conjugate of claim 9 wherein said at least 10 signal-generating groups are selected from the group consisting of an acridinium ester, an acridinium sulfonamide, and a mixture thereof and said hydrophilic carrier is BSA.

12. A method of detecting an analyte in a test sample comprising the steps of:
a. contacting a conjugate comprising: 1) at least 1-0 signal-generating groups; 2) an analyte-specific binding pair member; 3) a hydrophilic carrier and 4) a linker, with said test sample for a time and under conditions sufficient to form conjugate/analyte complexes; and
b. determining presence of said analyte in said test sample by detecting generation of a signal produced by said at least 10 signal-generating groups of said conjugate.

13. The method of claim 12 wherein said analyte is selected from the group consisting of: 1) a virus, a bacterium, a parasite and a fungus, 2) a fragment of said virus, bacterium, parasite or fungus, 3) an antibody to said virus, bacterium, parasite or fungus, and 4) a fragment of said antibody to said virus, bacterium, parasite and fungus.

14. The method of claim 12 wherein said hydrophilic carrier is selected from the group consisting of a polypeptide, a polysaccharide and a polyoxyethylene amine.

15. The method of claim 14 wherein said polypeptide is BSA.

16. The method of claim 12 wherein said at least 10 signal-generating groups are selected from the group consisting of a luminogen, a chemiluminogen, a chromogen, a fluorophore, a fluorogen, and a radioisotope.

17. The method of claim 16 wherein said luminogen is selected from the group consisting of an acridinium-containing compound, a phenanthridinium, a 1,2-dioxetane and luminol.

18. The method of claim 17 wherein said acridinium-containing compound is selected from the group consisting of an acridinium ester and an acridinium sulfonamide.

19. The method of claim 16 wherein said at least 10 signal-generating groups are luminogenic groups, wherein said at least 10 luminogenic groups may be the same or a mixture thereof.

20. The method of claim 12 wherein said analyte-specific binding pair member is selected from the group consisting of an antigen, an antibody, and a fragment of said antigen or said antibody having the same binding activity as said antigen or said antibody.

21. The method of claim 20 wherein said fragment of said antibody is selected from the group consisting of Fab, Fab′ and F(ab′)₂.

22. A method of detecting an analyte in a test sample comprising the steps of:
a. contacting said test sample with a first analyte-specific binding pair member for a time and under conditions sufficient to form analyte/first analyte-specific binding pair complexes;
b. contacting said resulting complexes of (a) with a conjugate comprising: 1) a hydrophilic carrier; 2) at least 10 signal-generating groups; 3) a second analyte-specific binding pair member and 4) a linker for a time and under conditions sufficient to form analyte/first analyte-specific binding pair member/conjugate complexes; and
c. determining the presence of said analyte in said test sample by detecting generation of a signal produced by said at least 10 signal-generating groups of said conjugate.

23. The method of claim 22 wherein said analyte is selected from the group consisting of: 1) a virus, a bacterium, a parasite and a fungus, 2) a fragment of said virus, bacterium or fungus, 3) an antibody to said virus, bacterium, parasite or fungus, and 4) a fragment of said virus, bacterium, parasite and fungus.

24. The method of claim 22 wherein said hydrophilic carrier is selected from the group consisting of a polypeptide, a polysaccharide and a polyoxyethylene amine.

25. The method of claim 24 wherein said polypeptide is BSA.

26. The method of claim 22 wherein said at least 10 signal-generating groups are selected from the group consisting of a luminogen, a chemiluminogen, a chromogen, a fluorophore, a fluorogen, and a radioisotope.

27. The method of claim 26 wherein said luminogen is selected from the group consisting of an acridinium-containing compound, a phenanthridinium, a 1,2-dioxetane and luminol.

28. The method of claim 27 wherein said acridinium-containing compound is selected from the group consisting of an acridinium ester and an acridinium sulfonamide.

29. The method of claim 26 wherein said at least 10 signal-generating groups are luminogenic groups, wherein said at least 10 luminogenic groups are the same or a mixture thereof.

30. The method of claim 22 wherein said analyte-specific binding pair member is selected from the group consisting of an antigen, an antibody, and a fragment of said antigen or said antibody having the same binding activity as said antigen or said antibody.

31. The method of claim 30 wherein said fragment of said antibody is selected from the group consisting of Fab, Fab′ and F(ab′)₂.

32. A kit for detection of an analyte in a test sample, wherein said kit comprises a conjugate, wherein said conjugate comprises: 1) a hydrophilic carrier, 2) at least 10 signal-generating groups, 3) an analyte-specific binding pair member and 4) a linker.

33. A method of forming a conjugate comprising the steps of:
a) incorporating into a hydrophilic carrier at least 10 signal-generating groups;
b) attaching a linker to said resulting product of step (a); and
c) conjugating an analyte-specific binding pair member to said attached linker of step (b) in order to form said conjugate.

34. The method of claim 33 wherein said hydrophilic carrier is selected from the group consisting of a polypeptide, a polysaccharide and a polyoxyethylene amine.

35. The method of claim 33 wherein said polypeptide is BSA.

36. The method of claim 33 wherein said at least 10 signal-generating groups are selected from the group consisting of a luminogen, a chromogen, a fluorophore, a fluorogen, and a radioisotope.

37. The method of claim 36 wherein said luminogen is selected from the group consisting of an acridinium-containing compound, a phenanthridinium, a 1,2-dioxetane and luminol.

38. The method of claim 37 wherein said acridinium-containing compound is selected from the group consisting of an acridinium ester and an acridinium sulfonamide.

39. The method of claim 36 wherein said at least 10 signal-generating groups are luminogenic groups, and said at least 10 luminogenic groups may be the same or a mixture thereof.

40. The method of claim 33 wherein said analyte-specific binding pair member is selected from the group consisting of an antigen, an antibody, and a fragment of said antigen or said antibody having the same binding activity as said antigen or said antibody.

41. The method of claim 40 wherein said fragment of said antibody is selected from the group consisting of Fab, Fab′ and F(ab′)₂
